(19) United States
(12) Patent Application Publication
(10) Pub. No.: US 2025/0129121 A1
(43) Pub. Date: Apr. 24, 2025

(54) POLYPEPTIDE, PRODUCT INCLUDING POLYPEPTIDE FOR INHIBITING FUSOBACTERIUM NUCLEATUM, OR DRUG FOR PREVENTING COLORECTAL CANCER

(71) Applicant: SUN YAT-SEN UNIVERSITY, Guangzhou (CN)

(72) Inventors: Wenjing Zhao, Guangzhou (CN); Shuli Chou, Guangzhou (CN); Xiangyu Mou, Guangzhou (CN)

(21) Appl. No.: 18/983,492

(22) Filed: Dec. 17, 2024

Related U.S. Application Data
(63) Continuation-in-part of application No. PCT/CN2023/100589, filed on Jun. 16, 2023.

(30) Foreign Application Priority Data
Jun. 17, 2022 (CN) 202210684148.6

(51) Int. Cl. C07K 7/06; A61K 35/00; A61K 38/00; A61P 31/04; C07K 7/08

(52) U.S. Cl. CPC C07K 7/06; A61K 35/00; A61P 31/04; C07K 7/08; A61K 38/00

(57) ABSTRACT

Provided are a polypeptide, a design method of the polypeptide, and a product including the polypeptide for inhibiting Fusobacterium nucleatum (F. nucleatum) or a drug for preventing colorectal cancer. The polypeptide includes a hydrophobic amino acid and a charged amino acid, where an amino acid sequence of the polypeptide is distributed in a symmetrical structure. The polypeptide obtained in the present application includes natural amino acid residues as basic ingredients, and has inherent biocompatibility and biodegradability. The polypeptide can specifically kill F. nucleatum while exhibiting a low antimicrobial activity against other bacteria. Therefore, the polypeptide can be used to restrict a load level of F. nucleatum in vivo for a long time, thereby achieving the treatment and prevention of a variety of F. nucleatum-associated diseases. In addition, the polypeptide has advantages such as high sterilization rate, no residue, no pollution, highly-flexible molecular composition, and low immunogenicity.

### CROSS-REFERENCE TO RELATED APPLICATIONS

The present application is a continuation-in-part application of PCT application No. PCT/CN2023/100589 filed on Jun. 16, 2023, which claims the benefit of Chinese Patent Application No. 202210684148.6 filed on Jun. 17, 2022. The contents of all of the aforementioned applications are incorporated by reference herein in their entirety.

### REFERENCE TO SEQUENCE LISTING

The Sequence Listing XML file submitted via the USPTO Patent Center, with a file name of “Sequence listing_SCH-24089-USCIP.xml”, a creation date of Dec. 17, 2024, and a size of 11,638 bytes, is part of the specification and is incorporated in its entirety by reference herein.

### TECHNICAL FIELD

The present application relates to the field of polypeptides, and in particular to a polypeptide and a design method thereof. The present application also relates to a product including the polypeptide for inhibiting Fusobacterium nucleatum (F. nucleatum) or a drug for preventing colorectal cancer.

### BACKGROUND

F. nucleatum is mainly found in the human oral cavity, and is a harmful commensal bacterium in humans. F. nucleatum is directly involved in oral diseases such as acute and chronic periodontitis, gingivitis, and root canal infections. It has also been found that F. nucleatum is associated with a range of diseases, including intestinal diseases such as colorectal cancer, inflammatory bowel disease, and appendicitis and diseases such as respiratory tract infections, adverse pregnancy outcomes, cardiovascular diseases, cerebral aneurysms, rheumatoid arthritis, and Alzheimer's disease.

F. nucleatum can be killed by broad-spectrum antibiotics, and acute F. nucleatum infection can be cured by antibiotics. However, antibiotics have the side effect of killing normal floras, and thus cannot be used for a long time. Once the administration of antibiotics is stopped, F. nucleatum, as a commensal bacterium in humans, can easily return to a high load level in the human body, which increases the risk of recurrence of acute and chronic infections and promotes the occurrence and development of the above F. nucleatum-associated diseases.

Therefore, a preparation that is harmless to the normal floras in the human body and can kill F. nucleatum is urgently required to restrict a load level of F. nucleatum in vivo for a long time, thereby achieving the treatment and prevention of the above various diseases.

### SUMMARY

In order to solve the above-mentioned technical problems, the present application provides a polypeptide that has an amino acid sequence symmetrically distributed and includes a hydrophobic amino acid and a charged amino acid in view of F. nucleatum. Some versions of the polypeptide have an antimicrobial activity against F. nucleatum, and some versions of the polypeptide exhibit a specific bactericidal effect for F. nucleatum. Since the polypeptide has a specific bactericidal effect for F. nucleatum and does not kill the normal floras of a host, the polypeptide can be used to reduce a load level of F. nucleatum in a host, thereby allowing the treatment or long-term prevention of F. nucleatum-associated diseases.

The present application provides a polypeptide, including a hydrophobic amino acid and a charged amino acid, where an amino acid sequence of the polypeptide is distributed in a symmetrical structure.

In some such solutions, the amino acid sequence of the polypeptide is symmetrically distributed at two sides of proline and glycine that serve as a center, and the proline and the glycine provide a β-turn, whereby the polypeptide has a β-fold structure.

In some such solutions, a total charge range of the polypeptide is 0 to +8.

In some such solutions, the charged amino acid includes a positively-charged amino acid and/or a negatively-charged amino acid; and the negatively-charged amino acid is glutamic acid and/or aspartic acid, and the positively-charged amino acid is at least one selected from the group consisting of histidine, lysine, and arginine.

In some such solutions, one or more oppositely-charged or uncharged amino acids are arranged between two adjacent charged amino acids with like charges.

In some such solutions, the hydrophobic amino acid is tryptophan.

In some such solutions, a proportion of the hydrophobic amino acid is 40% to 50%.

In some such solutions, a total length of the polypeptide is 10 to 20 amino acids.

In some such solutions, the amino acid sequence of the polypeptide is set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

In another aspect, the present application provides a design method for the polypeptide, including the following steps:
(1) fixing a type and content of the hydrophobic amino acid, designing an amino acid distribution into the symmetrical structure, and selecting a type and content of the charged amino acid; and
(2) producing a peptide resin with a polypeptide synthesizer through solid-phase chemical synthesis, and conducting TFA cleavage to produce the polypeptide.

The present application also provides a product including the polypeptide for inhibiting F. nucleatum or a drug including the polypeptide for preventing colorectal cancer.

In some such solutions, the polypeptide includes one or more selected from the group consisting of polypeptides with amino acid sequences set forth in SEQ ID NO: 6, SEQ ID NO: 7, SEQ ID NO: 8, SEQ ID NO: 9, SEQ ID NO: 10, SEQ ID NO: 11, and SEQ ID NO: 12, respectively. The polypeptide has an antibacterial effect for F. nucleatum. A bactericidal effect of the polypeptide is achieved by destroying a cell wall of F. nucleatum.

In some such solutions, the polypeptide includes a polypeptide with an amino acid sequence set forth in SEQ ID NO: 8 and/or SEQ ID NO: 11. The polypeptide has a specific antimicrobial activity against F. nucleatum, that is, the polypeptide dose not exhibit an antimicrobial activity or merely exhibits a low antimicrobial activity against test strains other than F. nucleatum.

In some such solutions, when the amino acid sequence of the polypeptide is set forth in SEQ ID NO: 8 or SEQ ID NO: 11, a dose of the polypeptide is 20 mg/kg, and an object of the polypeptide is a mouse.

Compared with the prior art, the embodiments of the present application have following beneficial effects:

Currently, in the clinical practice, the treatment of F. nucleatum infection mainly depends on antibiotics. However, antibiotics have the side effect of killing normal floras, and thus cannot be used for a long time. Once the administration of antibiotics is stopped, F. nucleatum, as a commensal bacterium in humans, can easily return to a high load level in the human body, which increases the risk of recurrence of acute and chronic infections and promotes the occurrence and development of various F. nucleatum-associated diseases. The polypeptide provided for F. nucleatum in the present application can specifically kill F. nucleatum while exhibiting a low antimicrobial activity against other bacteria, and thus does not have the side effect of antibiotics to kill normal floras. Therefore, the polypeptide can be used to restrict a load level of F. nucleatum in vivo for a long time, thereby achieving the treatment and prevention of a variety of F. nucleatum-associated diseases.

### BRIEF DESCRIPTION OF THE DRAWINGS

FIG. 1 shows an electrospray ionization mass spectrometry result of the polypeptide P7 obtained in Example 1 of the present disclosure;

FIG. 2 shows an electrospray ionization mass spectrometry result of the polypeptide P8 obtained in Example 1 of the present disclosure;

FIG. 3 shows an electrospray ionization mass spectrometry result of the polypeptide P9 obtained in Example 1 of the present disclosure;

FIG. 4 shows an electrospray ionization mass spectrometry result of the polypeptide P10 obtained in Example 1 of the present disclosure;

FIG. 5 shows an electrospray ionization mass spectrometry result of the polypeptide P11 obtained in Example 1 of the present disclosure;

FIG. 6 shows an electrospray ionization mass spectrometry result of the polypeptide P12 obtained in Example 1 of the present disclosure;

FIG. 7 shows a chemical molecular structure of the antimicrobial peptide P1 in the present application;

FIG. 8 shows a chemical molecular structure of the antimicrobial peptide P2 in the present application;

FIG. 9 shows a chemical molecular structure of the antimicrobial peptide P3 in the present application;

FIG. 10 shows a chemical molecular structure of the antimicrobial peptide P4 in the present application;

FIG. 11 shows a chemical molecular structure of the antimicrobial peptide P5 in the present application;

FIG. 12 shows a chemical molecular structure of the antimicrobial peptide P6 in the present application;

FIG. 13 shows a chemical molecular structure of the antimicrobial peptide P7 in the present application;

FIG. 14 shows a chemical molecular structure of the antimicrobial peptide P8 in the present application;

FIG. 15 shows a chemical molecular structure of the antimicrobial peptide P9 in the present application;

FIG. 16 shows a chemical molecular structure of the antimicrobial peptide P10 in the present application;

FIG. 17 shows a chemical molecular structure of the antimicrobial peptide P11 in the present application;

FIG. 18 shows a chemical molecular structure of the antimicrobial peptide P12 in the present application;

FIG. 19 shows transmission electron microscopy images of F. nucleatum treated with the polypeptides P7 to P12 and a blank group (Mock) in Example 3 of the present application, where a scale length for each image is 1 μm;

FIG. 20 is a schematic flow chart of an animal experiment in which F. nucleatum is treated with the polypeptides P8 and P11 in Example 5 of the present application; and

FIG. 21 shows statistical data of a tumor number (left) and a tumor size (right) of mice in Example 5 of the present application, where a significant difference (p<0.05) is marked with an asterisk (*), and a significant difference (p<0.01) is marked with asterisks (**).

### DETAILED DESCRIPTION OF THE EMBODIMENTS

The technical solutions in the embodiments of the present application are clearly and completely described below with reference to the accompanying drawings in the embodiments of the present application. Apparently, the described embodiments are merely some rather than all of the embodiments of the present application. All other embodiments obtained by those of ordinary skill in the art based on the embodiments of the present application without creative efforts should fall within the protection scope of the present application.

### Example 1 Design and Preparation of Polypeptides P1 to P12

In an aspect of design, the present application designs an amino acid distribution into a symmetrical structure to improve the antibacterial specificity. As a preferred solution, proline (P) and glycine (G) are used as a center to provide a β-turn, such that a polypeptide has a β-fold structure. The present application adopts a hydrophobic amino acid to increase the self-assembly potential of the polypeptide. As a preferred solution, tryptophan (W) is adopted as the hydrophobic amino acid. As a preferred solution, a proportion of the hydrophobic amino acid is designed to be 40% to 44%. In addition, the present application adopts a charged amino acid to adjust a total charge of the polypeptide, thereby regulating an interaction between the polypeptide and a target microorganism. As a preferred solution, a total charge range of the polypeptide is designed to be 0 to +8. As a preferred solution, charged amino acids are designed to be arranged alternately, and one or more oppositely-charged or uncharged amino acids are arranged between two adjacent charged amino acids with like charges. As a preferred solution, a total length of the polypeptide is designed to be 10 to 20 amino acids. As a preferred solution, amino acid sequences and physical and chemical parameters of the polypeptides P1 to P12 are shown in Table 1.

TABLE 1
Sequences and physical and chemical parameters of the polypeptides P1 to P12

| | | Hydrophobic | Hydro- | | Iso- | Total | |
|---|---|---|---|---|---|---|---|
| Poly- | Amino acid | amino acid | phobic | Hydro- | electric | charge | Linear |
| peptide | sequence | proportion | moment | phobicity | point | number | level |
| P1 | WEWRPGRWEW | 40% | 0.156 | 0.642 | 6.41 | 0 | 0.36 |
| | (SEQ ID NO: 1) | | | | | | |
| P2 | WEWRWEWRPGR | 44% | 0.119 | 0.673 | 7.00 | 0 | 0.21 |
| | WEWRWEW (SEQ | | | | | | |
| | ID NO: 2) | | | | | | |
| P3 | WEWRPGRWEW | 40% | 0.057 | 0.642 | 7.00 | 0 | 0.18 |
| | WEWRPGRWEW | | | | | | |
| | (SEQ ID NO: 3) | | | | | | |
| P4 | WDWRPGRWDW | 40% | 0.144 | 0.616 | 6.00 | 0 | 0.19 |
| | (SEQ ID NO: 4) | | | | | | |
| P5 | WDWRWDWRPG | 44% | 0.109 | 0.644 | 6.28 | 0 | 0.12 |
| | RWDWRWDW | | | | | | |
| | (SEQ ID NO: 5) | | | | | | |
| P6 | WDWRPGRWDW | 40% | 0.052 | 0.616 | 6.28 | 0 | 0.10 |
| | WDWRPGRWDW | | | | | | |
| | (SEQ ID NO: 6) | | | | | | |
| P7 | WHWRPGRWHW | 40% | 0.231 | 0.796 | 12.10 | 2 | 0.19 |
| | (SEQ ID NO: 7) | | | | | | |
| P8 | WHWRWHWRPG | 44% | 0.180 | 0.844 | 12.57 | 4 | 0.11 |
| | RWHWRWHW | | | | | | |
| | (SEQ ID NO: 8) | | | | | | |
| P9 | WHWRPGRWHW | 40% | 0.084 | 0.796 | 12.57 | 4 | 0.10 |
| | WHWRPGRWHW | | | | | | |
| | (SEQ ID NO: 9) | | | | | | |
| P10 | WKWRPGRWKW | 40% | 0.123 | 0.572 | 12.15 | 4 | 0.19 |
| | (SEQ ID NO: 10) | | | | | | |
| P11 | WKWRWKWRPG | 44% | 0.092 | 0.596 | 12.60 | 8 | 0.11 |
| | RWKWRWKW | | | | | | |
| | (SEQ ID NO: 11) | | | | | | |
| P12 | WKWRPGRWKW | 40% | 0.045 | 0.572 | 12.60 | 8 | 0.10 |
| | WKWRPGRWKW | | | | | | |
| | (SEQ ID NO: 12) | | | | | | |

In an aspect of preparation, the present application synthesizes the polypeptides P1 to P12 successively through solid-phase chemical synthesis. The polypeptide P1 is set forth in SEQ ID NO: 1, the polypeptide P2 is set forth in SEQ ID NO: 2, the polypeptide P3 is set forth in SEQ ID NO: 3, the polypeptide P4 is set forth in SEQ ID NO: 4, the polypeptide P5 is set forth in SEQ ID NO: 5, the polypeptide P6 is set forth in SEQ ID NO: 6, the polypeptide P7 is set forth in SEQ ID NO: 7, the polypeptide P8 is set forth in SEQ ID NO: 8, the polypeptide P9 is set forth in SEQ ID NO: 9, the polypeptide P10 is set forth in SEQ ID NO: 10, the polypeptide P11 is set forth in SEQ ID NO: 11, and the polypeptide P12 is set forth in SEQ ID NO: 12. The solid-phase chemical synthesis includes the following steps:
1. An amino acid sequence of each polypeptide was synthesized one by one from C terminus to N-terminus using a polypeptide synthesizer. Fmoc-X (X was a first amino acid at the C-terminus of each polypeptide) was introduced into a Wang resin, and then a Fmoc group was removed to produce an X-Wang resin. Then Fmoc-Y-(protection)-OH (9-fluorenylmethoxycarbonyl-trimethyl-Y, where Y was a second amino acid at the C-terminus of each antimicrobial peptide) was introduced. The protection sequences are shown in Table 2. According to the above procedure, each amino acid was introduced from C terminus to N-terminus to produce a peptide resin with a side chain protected and a Fmoc group removed.

TABLE 2
Protection sequences

| | Fmoc-Y-(protection)-OH |
|---|---|
| 1 | Fmoc-Ala-OH |
| 2 | Fmoc-Arg(Pbf)-OH |
| 3 | Fmoc-Asn(Trt)-OH |
| 4 | Fmoc-Arg(OtBu)-OH |
| 5 | Fmoc-Cys(Trt)-OH |
| 6 | Fmoc-Gln(Trt)-OH |
| 7 | Fmoc-Glu(OtBu)-OH |
| 8 | Fmoc-Gly-OH |
| 9 | Fmoc-His(Trt)-OH |
| 10 | Fmoc-Ile-OH |
| 11 | Fmoc-Leu-OH |
| 12 | Fmoc-Lys(Boc)-OH |
| 13 | Fmoc-Met-OH |
| 14 | Fmoc-Phe-OH |
| 15 | Fmoc-Pro-OH |
| 16 | Fmoc-Ser(tBu)-OH |
| 17 | Fmoc-Thr(tBu)-OH |
| 18 | Fmoc-Trp(Boc)-OH |
| 19 | Fmoc-Tyr(tBu)-OH |
| 20 | Fmoc-Val-OH |

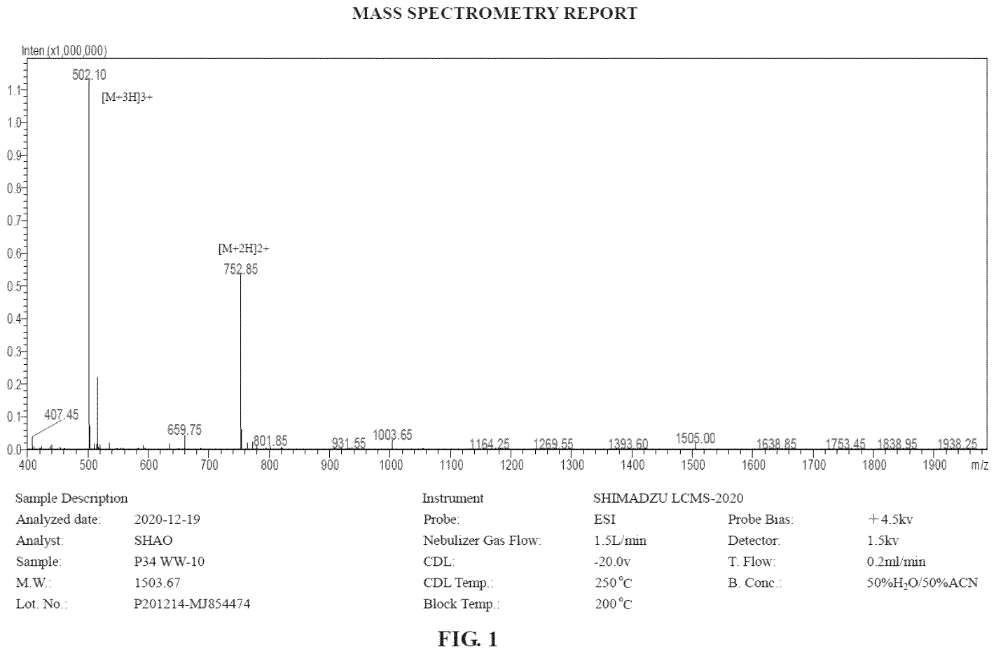

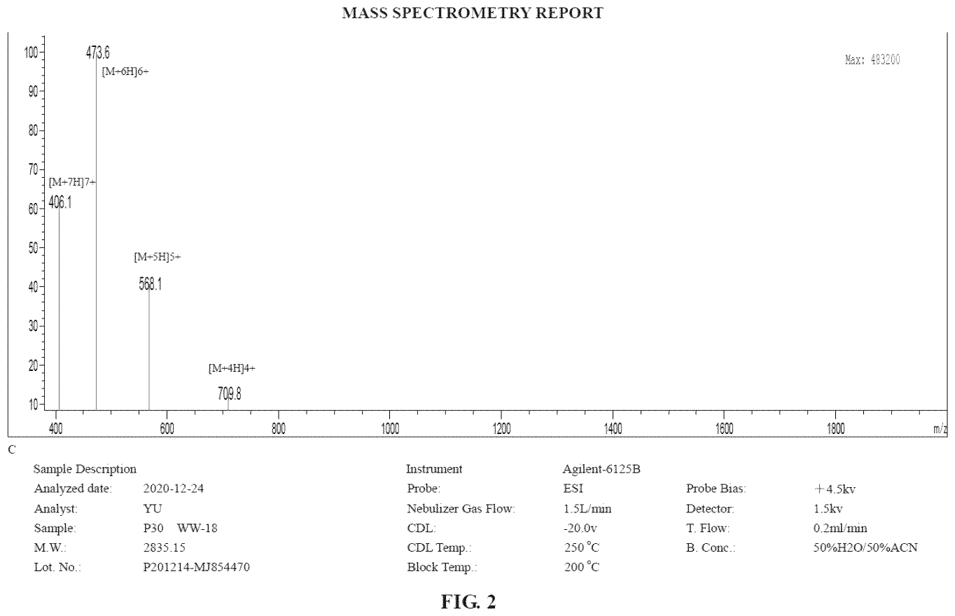

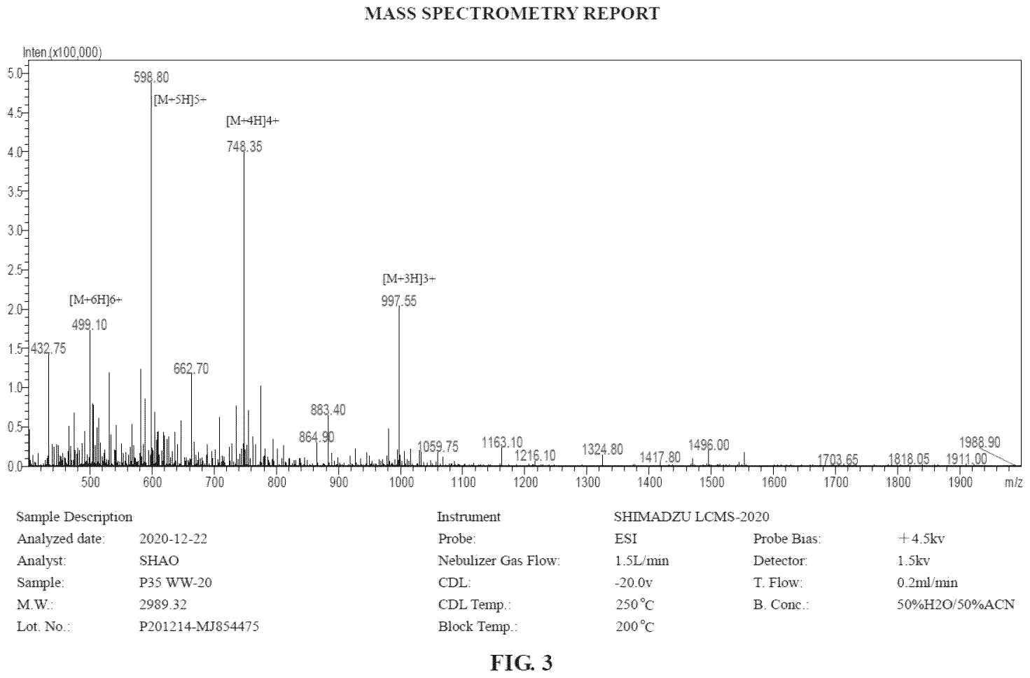

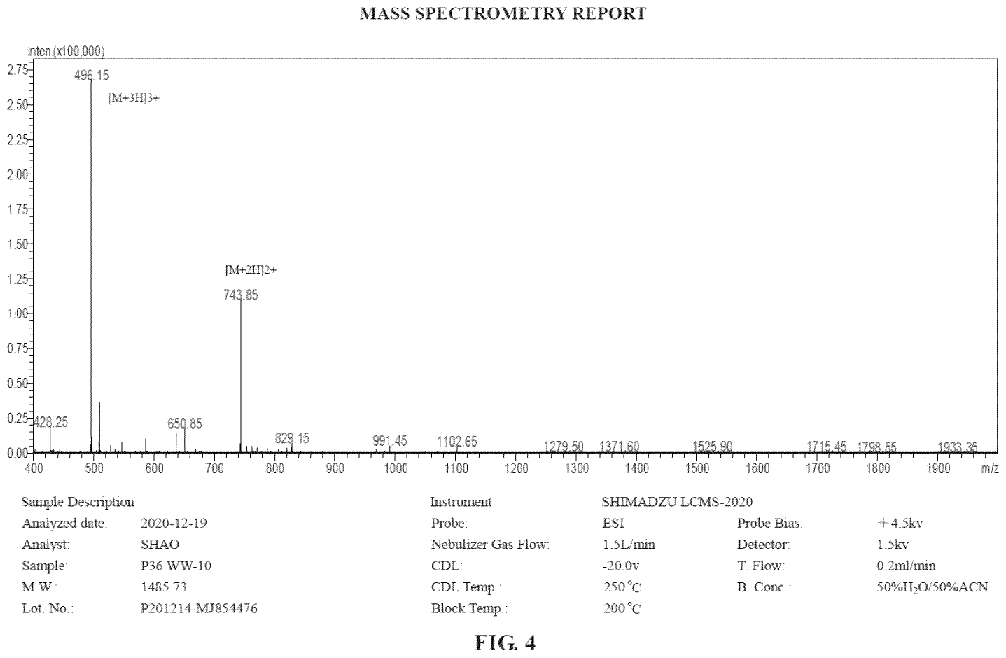

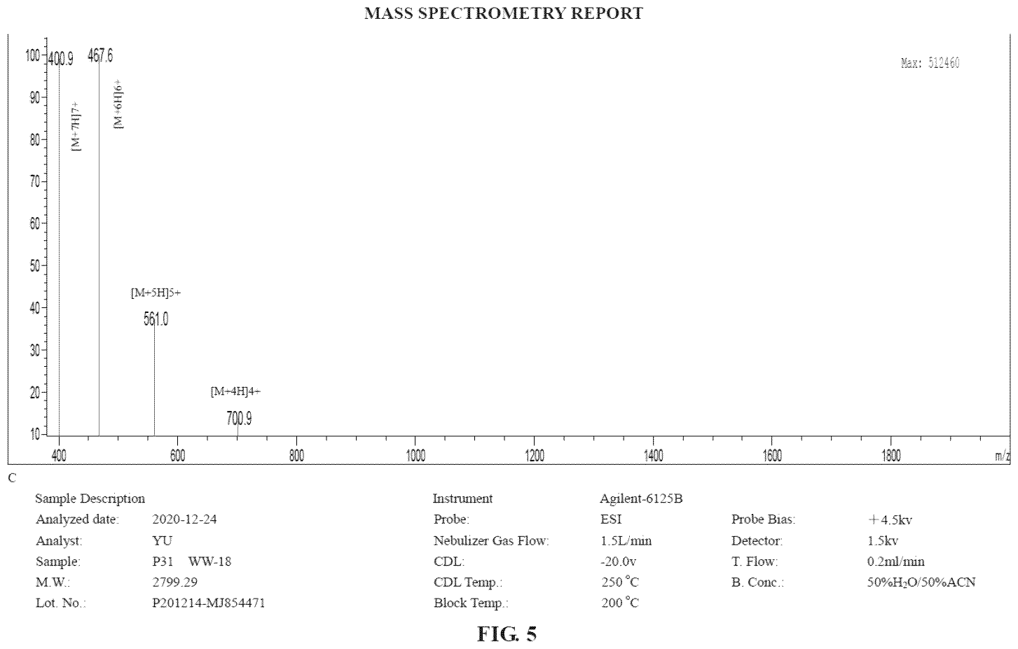

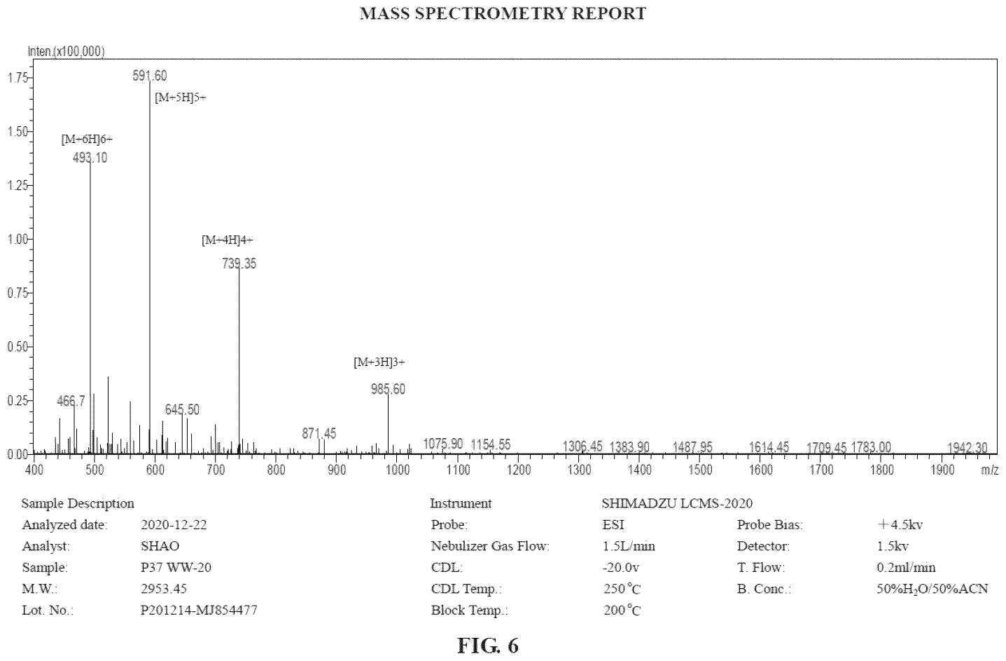

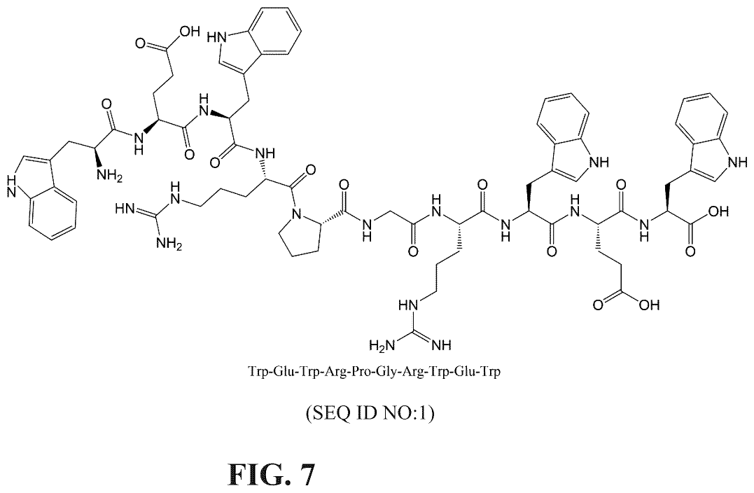

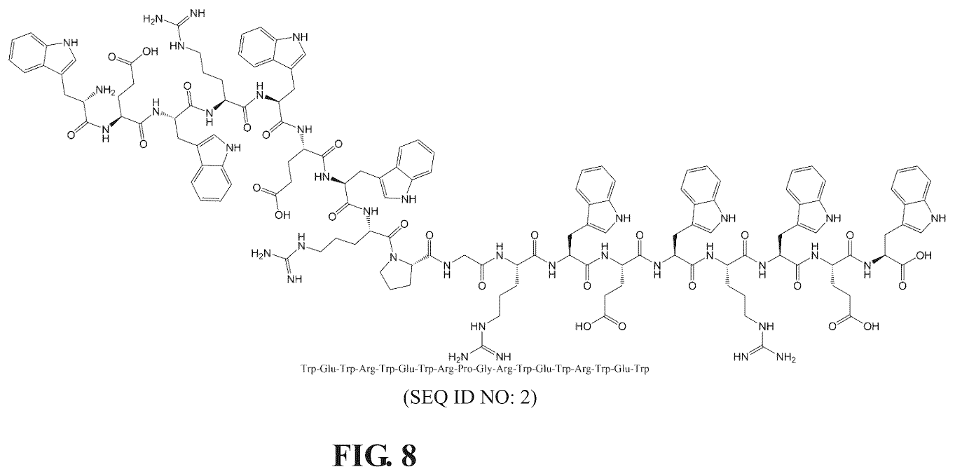

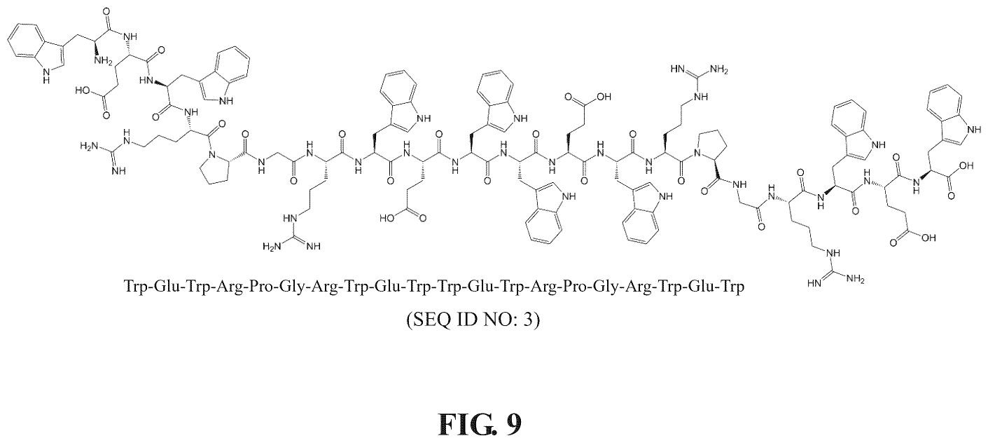

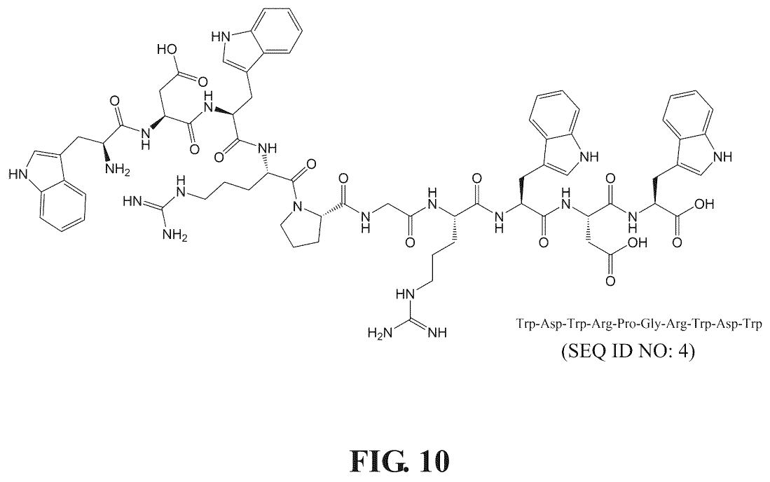

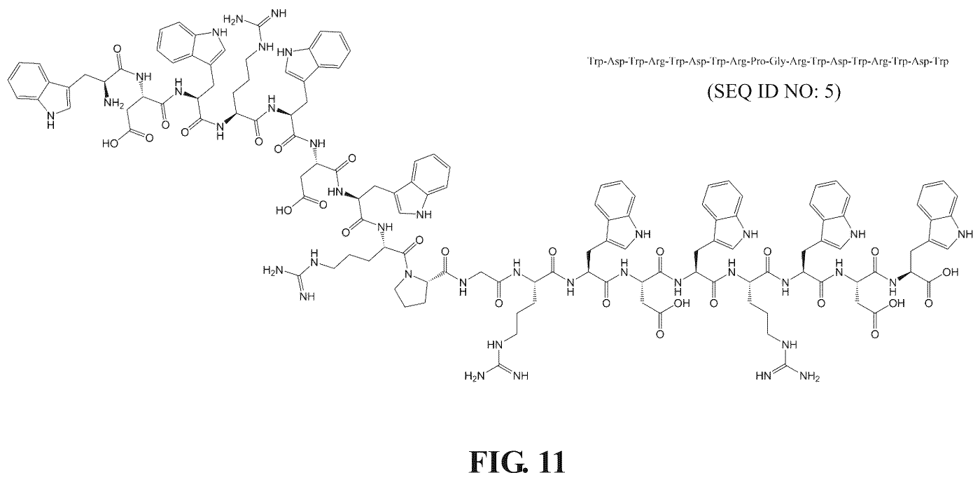

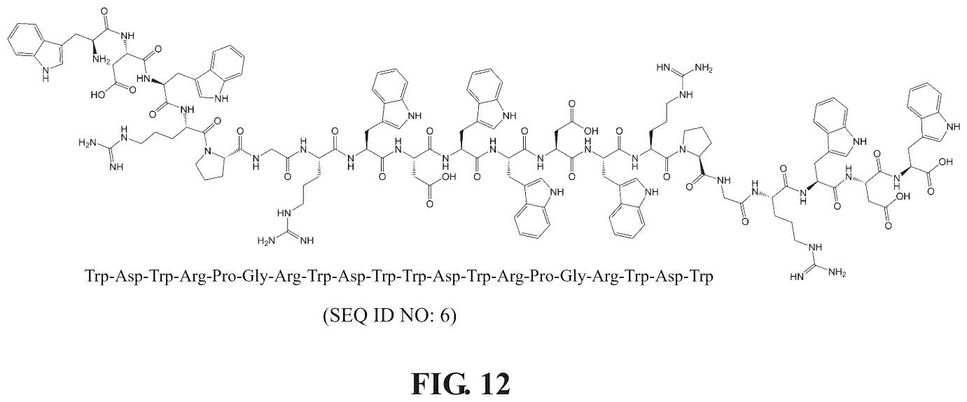

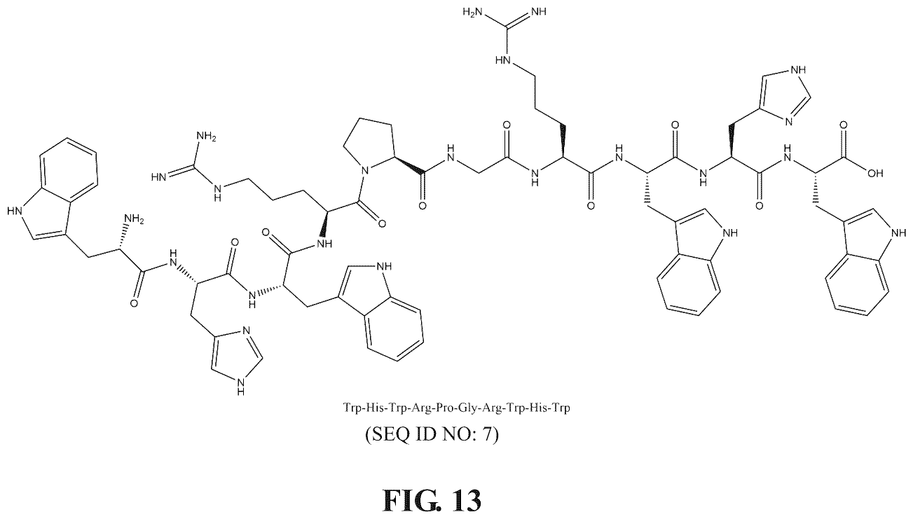

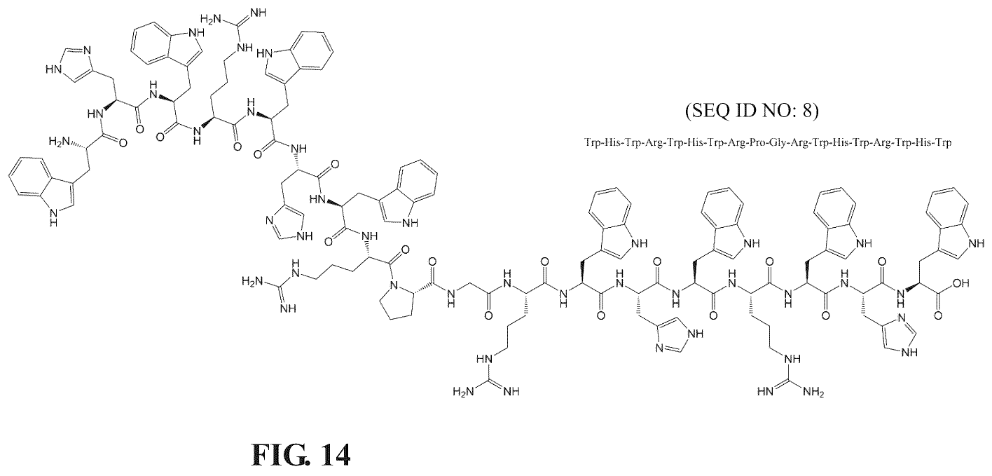

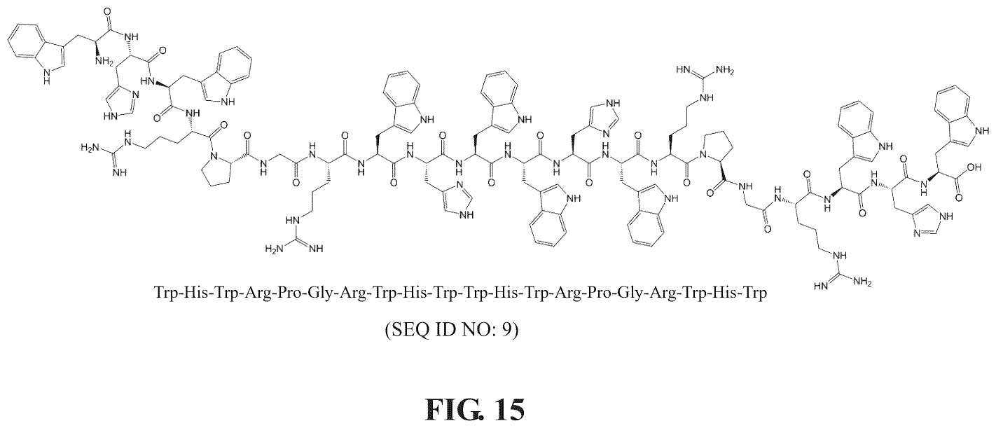

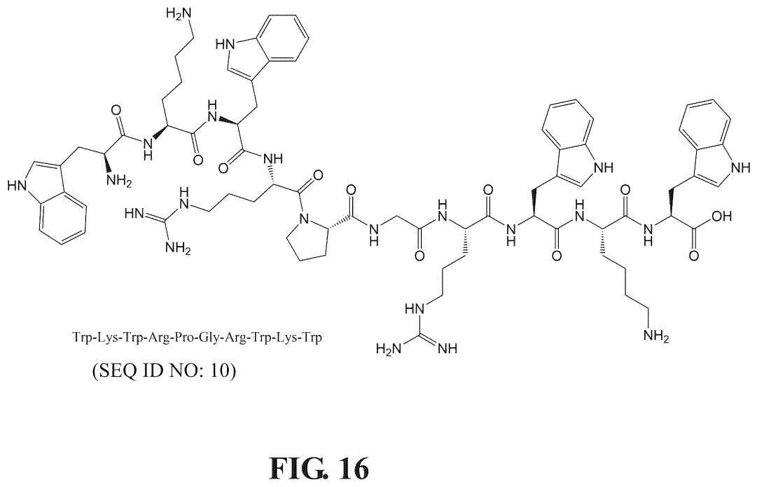

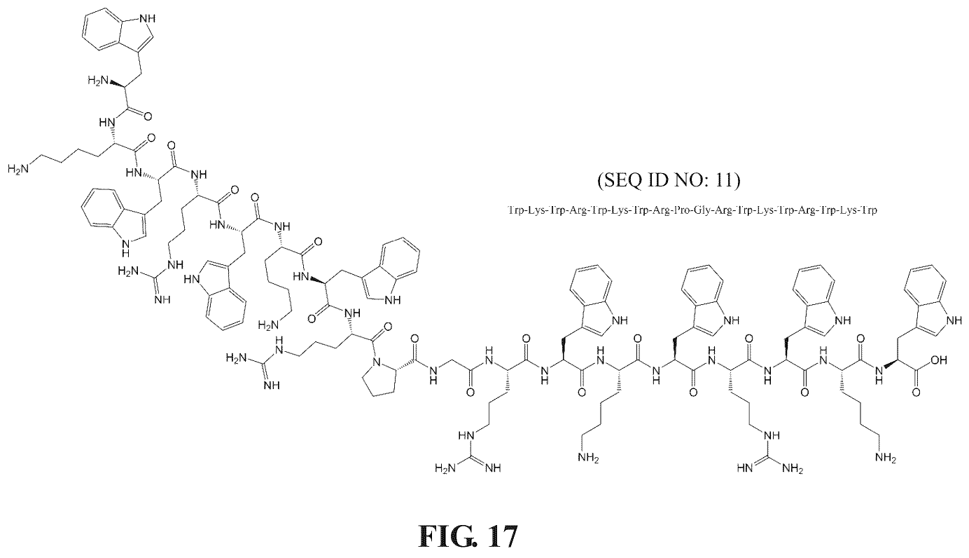

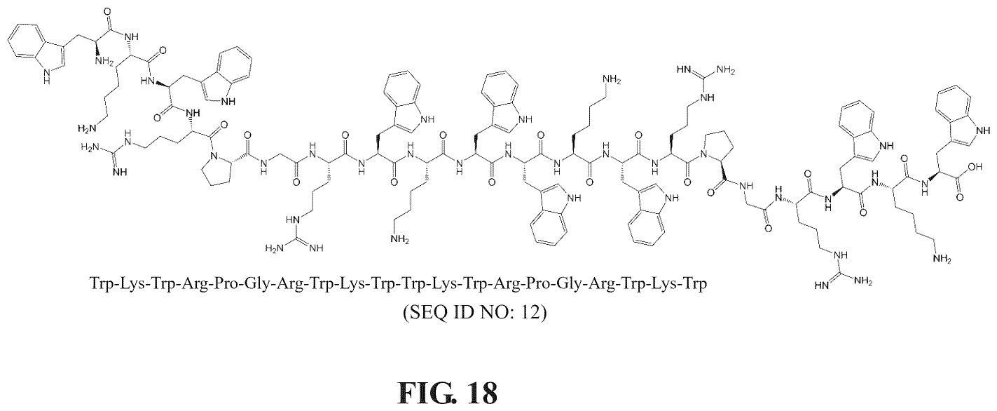

2. A cleavage reagent was added to the peptide resin, a reaction was conducted at 20° C. in the dark for 2 h, and filtration was conducted to produce a precipitate and a filtrate. The precipitate was washed with TFA to produce a washing solution. The washing solution was mixed with the filtrate to produce a mixed solution, and the mixed solution was concentrated with a rotary evaporator to produce a concentrate. Pre-cooled anhydrous diethyl ether was added in a volume about 10 times the volume of the concentrate, and precipitation was conducted at −20° C. for 3 h until a white powder was precipitated. Centrifugation was conducted at 2,500 g for 10 min to collect the white powder. Then, the white powder was washed with anhydrous diethyl ether and vacuum-dried to produce a polypeptide. The cleavage reagent was produced by mixing TFA, water, and triisopropyl chlorosilane (TIS) in a mass ratio of 95:2.5:2.5.
3. A column was equilibrated with 0.2 mol/L sodium sulfate (adjusting with phosphoric acid to a pH of 7.5) for 30 min. A polypeptide was dissolved with a 90% acetonitrile aqueous solution, filtered, and subjected to gradient elution with a C18 reversed-phase atmospheric column (an eluent was produced by mixing methanol and a sodium sulfate aqueous solution in a volume ratio of 30:70 to 70:30) with a flow rate of 1 mL/min and a detection wave of 220 nm. A main-peak product was collected, lyophilized, and further purified by a reversed-phase C18 column under the following conditions: an eluent A: 0.1% TFA/aqueous solution, an eluent B: 0.1% TFA/acetonitrile solution, an elution concentration: 25% B to 40% B, an elution time: 12 min, and a flow rate: 1 mL/min. A main-peak product was collected and lyophilized.
4. Identification of polypeptides: The polypeptides produced above each were analyzed by electrospray ionization mass spectrometry. Electrospray ionization mass spectrometry results of the polypeptides P7 to P12 are shown in FIG. 1 to FIG. 6, respectively. The electrospray ionization mass spectrometry results show that purities of the polypeptides P1 to P12 all are greater than 95%. Chemical structural formulas of the polypeptides P1 to P12 are shown in FIG. 7 to FIG. 18, respectively.

### Example 2 Determination of Antimicrobial Activities of the Polypeptides P1 to P12 in Vitro

1. Determination of antimicrobial activities:

A. Each polypeptide was prepared into a specified stock solution for later use. Minimum inhibitory concentrations (MICs) of the polypeptides P1 to P12 were determined by a broth microdilution method. With 0.01% acetic acid (including 0.2% of bovine serum albumin (BSA)) as a diluent, a series of gradient antimicrobial peptide solutions were prepared by a two-fold dilution method.

B. 100 μL of each polypeptide stock solution was taken and added to a 96-well cell culture plate, and then each strain solution to be tested (about 10⁵/mL) was added in an equal volume to each well. A positive control (including a strain solution, but not including a polypeptide) and a negative control (including neither a strain solution nor a polypeptide). Strains to be tested include Escherichia coli Nissile 1917, Vibrio cholerae H1, Pseudomonas aeruginosa PAO1, F. nucleatum 25586, F. nucleatum 10953, Bifidobacterium uniformis 6597, Lactobacillus acidophilus 6075, Bifidobacterium longum 6194Lactobacillus rhamnosus 6141, and Streptococcus agalactiae H94.

C. The cell culture plate was incubated at a constant temperature of 37° C. for 20 h, and MIC was recorded when the turbidity was not observed by naked eyes at a bottom of a well. Results are shown in Table 3. The strains all were purchased from the market.

TABLE 3
MICs of the polypeptides P1 to P12

| Strain | P1 | P2 | P3 | P4 | P5 | P6 | P7 | P8 | P9 | P10 | P11 | P12 |
|---|---|---|---|---|---|---|---|---|---|---|---|---|
| Escherichia coli | >64 | >64 | >64 | >64 | 64 | 64 | 64 | >64 | >64 | 64 | 64 | 32 |
| nissile1917 | | | | | | | | | | | | |
| (μM) | | | | | | | | | | | | |
| Vibrio cholerae | >64 | >64 | >64 | >64 | >64 | >64 | >64 | >64 | >64 | >64 | 64 | 64 |
| H1(μM) | | | | | | | | | | | | |
| Pseudomonas | >64 | >64 | >64 | >64 | >64 | >64 | >64 | >64 | >64 | >64 | 32 | 32 |
| aeruginosa | | | | | | | | | | | | |
| PAO1(μM) | | | | | | | | | | | | |
| Fusobacterium | >64 | >64 | >64 | >64 | >64 | 64 | 64 | 8 | 64 | 8 | 4 | 2 |
| nucleatum | | | | | | | | | | | | |
| 25586(μM) | | | | | | | | | | | | |
| Fusobacterium | >64 | >64 | >64 | >64 | >64 | 64 | 8 | 4 | 64 | 16 | 16 | 2 |
| nucleatum | | | | | | | | | | | | |
| 10953(μM) | | | | | | | | | | | | |
| Bifidobacteriu | >64 | >64 | >64 | >64 | >64 | >64 | >64 | >64 | >64 | 64 | 8 | 64 |
| muniformis | | | | | | | | | | | | |
| 6597(μM) | | | | | | | | | | | | |
| Lactobacillus | >64 | >64 | >64 | >64 | >64 | >64 | 32 | 32 | 64 | 64 | 32 | 32 |
| acidophilus 6075(μM) | | | | | | | | | | | | |
| Bifidobacterium longum | >64 | >64 | >64 | >64 | >64 | >64 | >64 | >64 | 64 | >64 | 16 | 16 |
| 6194(μM) | | | | | | | | | | | | |
| Lactobacterium | >64 | >64 | >64 | >64 | >64 | >64 | 64 | >64 | >64 | >64 | >64 | >64 |
| rhamnosus 6141(μM) | | | | | | | | | | | | |
| Streptococcus agalactiae | >64 | >64 | >64 | >64 | >64 | >64 | >64 | >64 | >64 | >64 | 64 | 2 |
| H94(μM) | | | | | | | | | | | | |

It can be seen from Table 2 that the polypeptides P6 to P12 have an antimicrobial activity against F. nucleatum (with MIC of less than or equal to 64 μM). Therefore, the polypeptides P6 to P12 can be referred to as antimicrobial peptides. It should be noted that the polypeptides P8 and P11 have a very strong antimicrobial activity against F. nucleatum (with MIC of merely 4 μM or 8 μM), but exhibit a weak antimicrobial activity against other strains, indicating obvious bactericidal specificity. Therefore, the polypeptides P8 and P11 can be called specific antimicrobial peptides for F. nucleatum.

### Example 3 Determination of an Antimicrobial Mechanism of the Polypeptides P7 to P12

A. Cells of F. nucleatum 25586 in a logarithmic growth phase were collected through centrifugation at 1,000 g for 5 min, rinsed 3 times with sterile phosphate-buffered saline (PBS), and resuspended to OD₆₀₀=0.2.

B. A polypeptide sample was added with a final concentration reaching 1×MIC to 40 mL of a strain solution prepared above, and the incubation was conducted on a shaker at 37° C. for 1 h.

C. A bacterial solution produced after the incubation was centrifuged at 5,000 g for 5 min to collect bacterial cells. The bacterial cells were rinsed three times with PBS, the buffer was completely removed, and then 500 μL of 2.5% glutaraldehyde was added immediately for resuspension to produce a bacterial suspension. The bacterial suspension was stored overnight at 4° C. in the dark.

D. A sample pre-fixed overnight as above was centrifuged to produce a precipitate. Then the precipitate was rinsed 3 times with sterile PBS to remove the residual pre-fixation solution. Osmic acid was added to allow post-fixation for 60 min to 120 min. Then a sample was centrifuged to produce a precipitate (because osmic acid is a biosafety-limit reagent, the removed osmic acid fixation solution should be carefully recovered). The precipitate was rinsed 3 times with sterile PBS to remove the residual post-fixation solution, treated for 8 min to 10 min with each of gradient ethanol solutions successively to allow gradient dehydration, then subjected to displacement for 10 min with each of 100% ethanol, a mixture of ethanol and acetone (1:1), and 100% tert-butanol successively, treated with a mixture of acetone and a resin (1:1) for 30 min, and then embedded with a pure resin overnight.

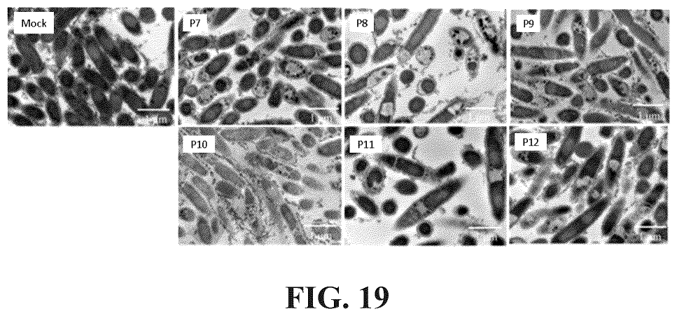

E. A sample was stained with uranyl acetate and lead citrate, prepared into ultrathin sections, and finally observed by a transmission electron microscope to determine changes in a membrane and an internal structure of a cell. Results are shown in FIG. 19.

It can be seen from the results in FIG. 19 that F. nucleatum cells treated with the polypeptides P7 to P12 undergo obvious cavities, plasmolysis, and cell membrane rupture, and the untreated cells are full and have intact cell wall structures. The results show that the polypeptides P7 to P12 act on a cell wall of F. nucleatum to cause a cell membrane rupture, thereby achieving the sterilization.

### Example 4 Determination of Self-Assemblies of the Polypeptides P7 to P12

The self-assembly capabilities of P7 to P12 were detected by dynamic light scattering: A polypeptide powder was dissolved, diluted to 32 μM, ultrasonically treated for 15 min, and then added to a vial. The vial was allowed to stand for 1 h, inserted into a vial holder, and placed for 10 min to 15 min until the sample had the same temperature as a hot bath, and then testing was conducted. Testing results show that the polypeptides P7 to P12 can form a self-assembled structure with a linearity of about 100 nm to 1,000 nm, which involves two orders of magnitude, where linearities of the polypeptides P8 and P11 exceed 1,000 nm.

### Example 5 Determination of in Vivo Activities of the Polypeptides P8 and P11

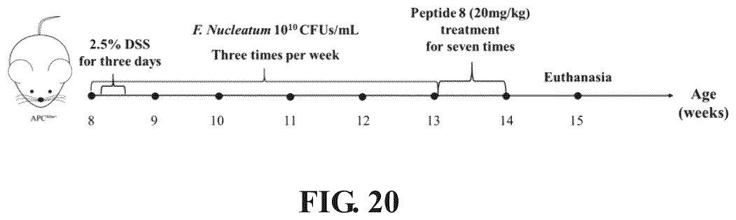

A. To verify the antibacterial effects of the F. nucleatum-specific antimicrobial peptides in vivo for F. nucleatum and especially the preventive effects of the F. nucleatum-specific antimicrobial peptides for F. nucleatum-induced diseases, an F. nucleatum-enhanced colorectal cancer animal model (i.e. the C57BL APC^{min} mouse orally administered with F. nucleatum) was adopted in this example. As shown in FIG. 20: C57BL APC^{min} mice were selected and intragastrically administered with F. nucleatum 25586 three times a week as an F. nucleatum intragastric administration group. Mice in a control group were intragastrically administered with an equal amount of PBS every week (PBS intragastric administration group). On the second day of intragastric administration in the first week, mice each were treated with 2.5% dextran sulfate sodium (DSS) consecutively for 3 d to improve the colonization of F. nucleatum.

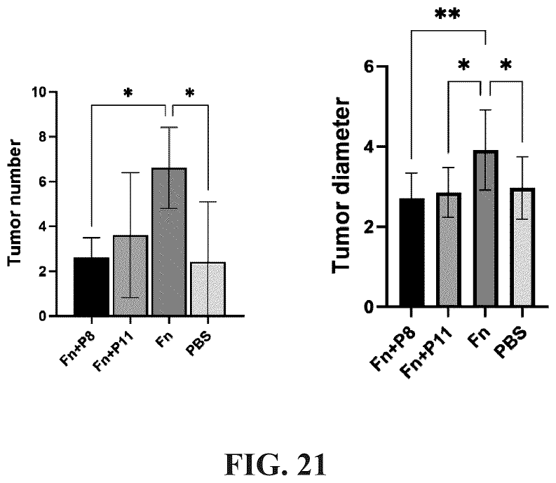

B. Mice were intragastrically administered with the antimicrobial peptide P8 or P11 for treatment in the sixth week, and mice in the control group were intragastrically administered with pure water. Each mouse was intragastrically administered at 20 mg/kg per day consecutively for 7 d. Then, mice were euthanized, and the tumor number and size were recorded. Results are shown in FIG. 21.

As shown in FIG. 21, compared with the F. nucleatum intragastric administration group intragastrically administered with pure water (Fn+H₂O), mice in the F. nucleatum intragastric administration groups intragastrically administered with the antimicrobial peptides P8 and P11 (Fn+P8 and Fn+P11) have a relatively small tumor number and a relatively small tumor diameter, and the tumor number and the tumor diameter are reduced to the levels of the PBS intragastric administration group without F. nucleatum intragastric administration. The experimental results show that the treatment with the antimicrobial peptides P8 and P11 has a preventive effect for F. nucleatum-induced colorectal cancer.

The objectives, technical solutions, and beneficial effects of the present application are further described in detail through the above specific examples. It should be understood that the above are merely some specific examples of the present application, but are not intended to limit the protection scope of the present application. It should be particularly noted that, any modifications, equivalent substitutions, improvements, etc. made by those skilled in the art within the spirit and principle of the present application should be included within the protection scope of the present application.

1. A polypeptide comprising a hydrophobic amino acid and a charged amino acid, wherein an amino acid sequence of the polypeptide is distributed in a symmetrical structure.

2. The polypeptide according to claim 1, wherein the amino acid sequence of the polypeptide is symmetrically distributed at two sides of proline and glycine that serve as a center, and the proline and the glycine provide a β-turn, whereby the polypeptide has a β-fold structure.

3. The polypeptide according to claim 1, wherein a total charge range of the polypeptide is 0 to +8.

4. The polypeptide according to claim 3, wherein the charged amino acid comprises a positively-charged amino acid and/or a negatively-charged amino acid; the negatively-charged amino acid is glutamic acid and/or aspartic acid, and the positively-charged amino acid is at least one selected from the group consisting of histidine, lysine, and arginine; and one or more oppositely-charged or uncharged amino acids are arranged between two adjacent charged amino acids with like charges.

5. The polypeptide according to claim 1, wherein a proportion of the hydrophobic amino acid is 40% to 50%, and the hydrophobic amino acid is tryptophan.

6. The polypeptide according to claim 1, wherein a total length of the polypeptide is 10 to 20 amino acids.

7. The polypeptide according to claim 1, wherein the amino acid sequence of the polypeptide is set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

8. The polypeptide according to claim 3, wherein the amino acid sequence of the polypeptide is set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

9. The polypeptide according to claim 7, wherein the amino acid sequence of the polypeptide is set forth in SEQ ID NO: 8 or SEQ ID NO: 11.

10. A design method for the polypeptide according to claim 1, comprising the following steps:
(1) fixing a type and content of the hydrophobic amino acid, designing an amino acid distribution into the symmetrical structure, and selecting a type and content of the charged amino acid; and
(2) producing a peptide resin with a polypeptide synthesizer through solid-phase chemical synthesis, and conducting trifluoroacetic acid (TFA) cleavage to produce the polypeptide.

11. A design method for the polypeptide according to claim 2, comprising the following steps:
(1) fixing a type and content of the hydrophobic amino acid, designing an amino acid distribution into the symmetrical structure, and selecting a type and content of the charged amino acid; and
(2) producing a peptide resin with a polypeptide synthesizer through solid-phase chemical synthesis, and conducting TFA cleavage to produce the polypeptide.

12. A design method for the polypeptide according to claim 7, comprising the following steps:
(1) fixing a type and content of the hydrophobic amino acid, designing an amino acid distribution into the symmetrical structure, and selecting a type and content of the charged amino acid; and
(2) producing a peptide resin with a polypeptide synthesizer through solid-phase chemical synthesis, and conducting TFA cleavage to produce the polypeptide.

13. A product for inhibiting Fusobacterium nucleatum (F. nucleatum) or a drug for preventing colorectal cancer, comprising the polypeptide according to claim 1.

14. A product for inhibiting F. nucleatum or a drug for preventing colorectal cancer, comprising the polypeptide according to claim 2.

15. A product for inhibiting F. nucleatum or a drug for preventing colorectal cancer, comprising the polypeptide according to claim 3.

16. A product for inhibiting F. nucleatum or a drug for preventing colorectal cancer, comprising the polypeptide according to claim 4.

17. A product for inhibiting F. nucleatum or a drug for preventing colorectal cancer, comprising the polypeptide according to claim 5.

18. A product for inhibiting F. nucleatum or a drug for preventing colorectal cancer, comprising the polypeptide according to claim 6.

19. A product for inhibiting F. nucleatum or a drug for preventing colorectal cancer, comprising the polypeptide according to claim 7.

20. The product for inhibiting F. nucleatum or the drug for preventing colorectal cancer according to claim 13, wherein the polypeptide comprises one or more selected from the group consisting of polypeptides with amino acid sequences set forth in SEQ ID NO: 6, SEQ ID NO: 7, SEQ ID NO: 8, SEQ ID NO: 9, SEQ ID NO: 10, SEQ ID NO: 11, and SEQ ID NO: 12.


SEQUENCE LISTING (12 sequences)

SEQ ID NO: 1 - protein, 10 aa, synthetic construct
WEWRPGRWEW

SEQ ID NO: 2 - protein, 18 aa, synthetic construct
WEWRWEWRPGRWEWRWEW

SEQ ID NO: 3 - protein, 20 aa, synthetic construct
WEWRPGRWEWWEWRPGRWEW

SEQ ID NO: 4 - protein, 10 aa, synthetic construct
WDWRPGRWDW

SEQ ID NO: 5 - protein, 18 aa, synthetic construct
WDWRWDWRPGRWDWRWDW

SEQ ID NO: 6 - protein, 20 aa, synthetic construct
WDWRPGRWDWWDWRPGRWDW

SEQ ID NO: 7 - protein, 10 aa, synthetic construct
WHWRPGRWHW

SEQ ID NO: 8 - protein, 18 aa, synthetic construct
WHWRWHWRPGRWHWRWHW

SEQ ID NO: 9 - protein, 20 aa, synthetic construct
WHWRPGRWHWWHWRPGRWHW

SEQ ID NO: 10 - protein, 10 aa, synthetic construct
WKWRPGRWKW

SEQ ID NO: 11 - protein, 18 aa, synthetic construct
WKWRWKWRPGRWKWRWKW

SEQ ID NO: 12 - protein, 20 aa, synthetic construct
WKWRPGRWKWWKWRPGRWKW